(19) United States
(12) Patent Application Publication
(10) Pub. No.: US 2024/0272174 A1
(43) Pub. Date: Aug. 15, 2024

(54) IMMUNOLOGICAL METHODS INVOLVING DRA:DQB AND DPA:DQB HETERODIMERS

(71) Applicant: THE TERASAKI INNOVATION CENTER, Glendale, CA (US)

(72) Inventors: Neng Jen Remi Shih, Winnetka, CA (US); Thoa Nong, Granada Hills, CA (US); Jar-How Lee, Los Angeles, CA (US)

(21) Appl. No.: 18/435,898

(22) Filed: Feb. 7, 2024

Related U.S. Application Data
(60) Provisional application No. 63/444,406, filed on Feb. 9, 2023.

(51) Int. Cl. G01N 33/68; G01N 33/58

(52) U.S. Cl. CPC G01N 33/6854; G01N 33/58

(57) ABSTRACT

Described herein are materials and methods of screening transplant donors and recipients for the presence of antibodies specific for DRα:DQβand DPα:DQβ heterodimer antigens.

### BACKGROUND

Transplant rejection occurs when the immune system of the recipient of a transplant, particularly antibodies produced by the recipient, attacks the transplanted organ or tissue. The recipient's immune system recognizes the transplanted organ as foreign tissue and attempts to destroy it (host vs graft reaction). Rejection also occurs when the transplanted organ consists of donor lymphocytes or progenitor stem cells, which may generate an immune response to the recipient tissues (graft vs. host disease). Chronic rejection is a term used to describe all long-term loss of function in organ transplants associated with chronic alloreactive immune response. Chronic rejection usually leads to graft failure and a need for a new transplanted organ about a decade after the initial transplant. Human leukocyte antigens (HLA) are one type of target molecule within a transplanted organ to which the recipient's immune system responds resulting in transplant rejection. The HLA system is highly polymorphic in nature.

It is a standard practice in the transplant field to evaluate all potential recipients for HLA antibodies by testing against a panel of HLA antigens selected to represent an approximate distribution in the human population. In vitro testing determines the specificity of HLA antibodies present in a patient's blood. The detection of HLA antibodies in a patient's serum that are directed against specific HLA alleles present in a potential donor (donor-specific antibody; DSA) is an indication of risk for graft rejection or failure.

Alloantibodies, particularly when donor specific, are one of the most important factors that cause both early and late graft rejection. Despite improvements in transplantation outcomes, antibody-mediated rejection (AMR) remains a significant impediment to long term graft survival and is associated with increased morbidity, mortality and costs (Colvin, ASN 18(4):1046-1056, 2007).

The presence of HLA antibody is widely believed to be one of the major elements contributing to humoral graft rejections. Transplant recipients with high levels of DSA are at risk for with early graft rejection and failure. Elevated donor specific HLA antibodies in organ recipients either before and/or after allograft transplantation has been associated with acute and chronic AMR and decreased long term graft survival.

HLA molecules exist in two forms; HLA class I molecules consist of a 45-kDa glycoprotein (heavy chain) non covalently associated with a 12-kDa polypeptide, β2-microglobulin (β2m). Association of β2m with newly synthesized class I heavy chains is required in order for the HLA molecule to be expressed and present the peptide (Krangel et al., Cell 18:979, 1979). However, β2m free class I heavy chains were identified on activated T lymphocytes (Schnabl et al., J. Exp. Med. 171:1431, 1990) and other cell surfaces (Bix & Raulet, J. Exp. Med. 176(3) 829-34, 1992).

HLA class Il molecules are heterodimers formed by noncovalent association of two glycosylated polypeptide chains referred to as alpha and beta chains. The a subunit is 33 kDa and the β subunit is 28 kDa, and both chains are transmembrane polypeptides that have the same overall structure. The non-polymorphic a chain is encoded by the DRA gene and can associate with various polymorphic β chains that are encoded by a specific DRB gene. In addition, the DP and DQ HLA gene families each have one gene that encodes an a chain and a β chain. (Reviewed in Choo, Yonsei Med. J. 48:11-23, 2007).

The Human Leukocyte Antigen (HLA) gene cluster is located within the Major Histocompatibility Complex (MHC) region on the short arm of human chromosome 6. HLA Class I molecules are comprised of a heavy chain and a 32 microglobulin, while HLA Class II molecules are assembled by the α and β chains. However, both types of molecules have similar tertiary structure with the two α helix sidewalls and a β sheet creating a natural grove to bind peptides from both self and non-self-antigens.

The HLA system contains the most polymorphic gene cluster in the entire human genome and is located on the short arm of human chromosome 6. Currently there are 25,019 Class I alleles and 10,201 Class II HLA alleles as reported by the IMGT/HLA Database. The different alleles account for a great deal of diversity of HLA antigens to present as many peptides as possible to TCR's and elicit a specific immune response against most antigens. Due to their polymorphic nature HLA antigens can serve as immunogens and elicit a cellular and humoral alloimmune responses as well. The most common mechanisms of inducing alloimmunity are via pregnancies, transfusions and transplants. Among transplant recipients, an alloantibody response to a donor's HLA antigens has been shown to be a cause of allograft failure. (Patel and Terasaki, NEJM 280:735, 1969) Pre-transplant, candidates are routinely screened for the presence of Class I and Class Il antibodies with solid phase assays utilizing beads coated with single HLA proteins. Normally, DRβ chains pair with a specific, and minimally polymorphic, DRα chain. The same is true for DQ and DP alpha and beta chains, however, in contrast to DRα, both DQA and DPA are highly polymorphic. Importantly, further diversity is generated when DRα pairs with DQβ forming unique, cross-locus heterodimers.

DRA and DQβ heterodimers have been previously described but they have been deemed unstable and there is no evidence that shows that such hybrid molecules are assembled, in vivo into functional and stable dimes capable of eliciting an immune response. Lotteau et al. (Nature 1987; 329:339-341) This heterodimer has been found in a transformed B cell lines that express high levels of the DRa chain. Modulation of the level of expression of the DRα chains in B cells lines controlled the formation of the DRα/DQβ pair, in that only cells expressing high levels of DRA mRNA produced this mixed heterodimers (J Exp. Med. 1989;169:351-356) suggesting an inefficient assembly of the heterodimer pair. Karp et al. (J. Exp. Med. 1990;171:615-628) attempted to address whether mixed heterodimers of human class Il form intracellularly and whether they are expressed on the cell surface and which parts of the Class II β chains are important for the ability to pair with α chains.

Mixed isotype class Il molecules have also been examined in both murine and human systems. Long et al. (Molecular Biology of HLA Class | Antigens. J. Silver, ed. CRC Press, Inc., Boca Raton, 1990. pp. 65-84.) and Karp, et al. J. Exp. Med. 171:615 1990. demonstrated the surface expression of mixed isotype heterodimers by transfecting various HLA class Il cDNA into mouse fibroblasts and COS cells, respectively. In addiiton Lotteau et al, (Nature 1987;329:339-341) detected DRα/DQβ dimers in EBV-transformed B-LCL by immunoprecipitation. Several studies with mice indicate that allelic variation of the Aβ chain influences it's a bility to form surface heterodimers with the a chain. According to earlier studies allelic variations of the DQβ molecules markedly influenced surface heterodimer formation with DRa molecules. Previous studies suggested that the DQw1β chain probably did not have a high affinity for the DRachain, or the DRα/DQ1βdimer is subjected to abnormal intracellular trafficking in reaching the cell surface, inasmuch as introduction of the DQw1β cDNA into either a homozygous DQw2 or DQw3 B-LCL gave very low surface expression of the DQw1 molecule. This observation was said to suggest that the DQw1β chain for binding of the DRa chain or with the endogenous DQw2β or DQw3β chains for binding to the DQα chains in those cells (Kwok WW, Kovats S, Thurtle P, Nepom GT. HLA-DQ allelic polymorphisms constrain patterns of class Il heterodimer formation. J Immunol. 1993; 150:2263-2272). Lotteau et al. was also said to sugges that the inracellular pool of DRa peptide would influence the amount of immunoprecipitable DRα/DQβ complex.

### SUMMARY

The invention relates to the existence and immunogenicity of stable DRα:DQβ heterodimers in both an EBV transformed B cell line and in transfectants constructed by co-transfection of DQB1 and DRA cDNA fragments. Some DQB1 alleles, especially those in the DQ1 serological group, appear to be more efficient in forming cell surface expressed DRα:DQβ heterodimers.

The cross-locus combination of DRα with DQβ generates a new class of peptide binding motifs not previously appreciated, thereby expanding the repertoire of bound peptides and further expanding the possibility of complex alloreactive immune responses.

Disclosed herein is the assembly of DQβ1 and DRα hybrids and of DQβ1 and DRα hybrids in the B cell lines and the successful isolation of the hybrid proteins in recombinant human cell lines. Transplant patients are also shown to produce allo-antibodies to these hybrid antigens.

According to one aspect of the invention the immunogenicity of DQβ0601:DRα and DQβ0603:DRa heterodimers was investigated and DQβ0601:DRa specific antibodies were detected in 6% of the transplant candidates on the UNOS waiting list while DQβ0603:DRα antibodies were detected but in fewer cases within the same cohort.

The existence of such antibodies is potentially problematic in transplantation and transfusion because of the potential for the occurrence of graft versus host and host versus graft reactions in the transplantation recipient. The invention therefore provides methods for the detection of antibodies that bind to a DRα:DQβ heterodimer in biological samples.

According to one aspect of the invention a method is provided of screening a biological sample for antibodies that bind to a DRα:DQβ heterodimer comprising the steps of:obtaining a biological sample from a subject, contacting the biological sample with a DRα:DQβ heterodimer, and detecting the binding of antibodies in said sample to the DRα:DQβ heterodimer. According to one aspect of the invention the subject is a mammalian subject and more preferably a human subject.

The method can be practiced by any of a variety of methods know to those in the art but according to one preferred method binding of an antibody is detected using flow cytometry. According to one method antibody binding is detected with a secondary antibody and the secondary antibody can comprise a label selected from the group consisting of a radioactive label, fluorescent label, enzymatic label, colorimetric label avidin label and biotin label.

The methods are particularly useful in determining compatibility of donors and recipients for tissue transplantation including transfusions and the human subject is a transplant or transfusion recipient or a transplant or transfusion donor.

According to alternative aspects of the invention a method is provided for screening a biological sample for the presence of DRα:DQβ heterodimer antigens comprising the steps of:obtaining a biological sample from a subject, contacting the biological sample with an antibody specific for said DRα:DQβ heterodimer, and detecting the binding of said antibodies in said sample to a DRα:DQβ heterodimer.

According to such methods the subject is preferably a mammal and more preferably a human subject.

The method can be practiced by any of a variety of methods know to those in the art but according to one preferred method binding of an antibody is detected using flow cytometry. According to one method antibody binding is detected with a secondary antibody and the secondary antibody can comprise a label selected from the group consisting of a radioactive label, fluorescent label, enzymatic label, colorimetric label avidin label and biotin label.

The methods are particularly useful in determining compatibility of donors and recipients for tissue transplantation including transfusions and the human subject is a transplant or transfusion recipient or a transplant or transfusion donor.

More specifically are provided methods of determining whether a donor tissue intended for transplant or transfusion is suitable for transplantation or transfusion to a human recipient expressing a DRα:DQβ heterodimer comprising a) obtaining a sample of the tissue intended for transplant or transfusion in the human subject, b) contacting the sample with a DRα:DQβ heterodimer, and c) detecting the complex of a DRα:DQβ heterodimer and antibody specific for a DRα:DQβ heterodimer, wherein the presence of the complex indicates that the donor tissue is unsuitable for transplantation to the human recipient. In this case, the donor tissue containing antibodies against the DRα:DQβ heterodimer are unsuitable to recipients having the DRα:DQβ heterodimer.

Methods of determining whether a donor tissue intended for transplant or transfusion is suitable for transplantation or transfusion to a human recipient having antibodies to a DRα:DQβ heterodimer comprising a) obtaining a sample of the tissue intended for transplant or transfusion in the human subject, b) contacting the sample with an antibody specific for DRα:DQβ heterodimer, and c) detecting the complex of a DRα:DQβ heterodimer and antibody specific for a DRα:DQβ heterodimer, wherein the presence of the complex indicates that the donor tissue is unsuitable for transplantation to the human recipient. In this case, the donor tissue containing DRa:DQβ heterodimer are unsuitable for recipients having antibodies to the DRα:DQβ heterodimer.

Also provided are kits for screening for antibodies that specifically bind to a DRα:DQβ heterodimer comprising a panel of solid-phase substrates in which a DRα:DQβ heterodimer is immobilized thereto. According to one embodiment kits are provided wherein the solid-phase substrate is a set of microparticles wherein each microparticle presents at least one DRα:DQβ heterodimer antigen. According to a further aspect, kits are provided in which the solid phase substrate is a single antigen bead.

Also provided are kits for screening for DRα:DQβ heterodimer antigens comprising a panel of antibodies specific for a DRα:DQβ heterodimer. According to one embodiment the solid-phase substrate is a set of microparticles wherein each microparticle presents at least one DRα:DQβ heterodimer antibody. According to a further embodiment the solid phase substrate is a single antibody bead.

As a further aspect of the invention the existence of naturally occurring antibodies specific for stable DQβ:DPα heterodimers is described. The occurrence of such naturally occurring antibodies to DQβ:DPα antigens is similarly problematic in human transplantation and transfusion because of the potential for the occurrence of graft versus host and host versus graft reactions in transplant recipients.

According to one aspect of the invention a method is provided of screening a biological sample for antibodies that bind to a DPα:DQβ heterodimer comprising the steps of:obtaining a biological sample from a subject, contacting the biological sample with a DPα:DQβ heterodimer, and detecting the binding of antibodies in said sample to the DPα:DQβ heterodimer. According to one aspect of the invention the subject is a mammalian subject and more preferably a human subject.

The method can be practiced by any of a variety of methods know to those in the art but according to one preferred method binding of an antibody is detected using flow cytometry. According to one method antibody binding is detected with a secondary antibody and the secondary antibody can comprise a label selected from the group consisting of a radioactive label, fluorescent label, enzymatic label, colorimetric label avidin label and biotin label.

The methods are particularly useful in determining compatibility of donors and recipients for tissue transplantation including transfusions and the human subject is a transplant or transfusion recipient or a transplant or transfusion donor.

According to alternative aspects of the invention a method is provided for screening a biological sample for the presence of DPα:DQβ heterodimer antigens comprising the steps of:obtaining a biological sample from a subject, contacting the biological sample with an antibody specific for said DPα:DQβ heterodimer, and detecting the binding of said antibodies in said sample to a DPα:DQβ heterodimer.

According to such methods the subject is preferably a mammal and more preferably a human subject.

The method can be practiced by any of a variety of methods known to those in the art but according to one preferred method binding of an antibody is detected using flow cytometry. According to one method antibody binding is detected with a secondary antibody and the secondary antibody can comprise a label selected from the group consisting of a radioactive label, fluorescent label, enzymatic label, colorimetric label avidin label and biotin label.

The methods are particularly useful in determining compatibility of donors and recipients for tissue transplantation including transfusions and the human subject is a transplant or transfusion recipient or a transplant or transfusion donor.

More specifically are provided methods of determining whether a donor tissue intended for transplant or transfusion is suitable for transplantation or transfusion to a human recipient expressing a DPα:DQβ heterodimer comprising a) obtaining a sample of the tissue intended for transplant or transfusion in the human subject, b) contacting the sample with a DPα:DQβ heterodimer, and c) detecting the complex of a DPα:DQβ heterodimer and antibody specific for a DPα:DQβ heterodimer, wherein the presence of the complex indicates that the donor tissue is unsuitable for transplantation to the human recipient. In this case, the donor tissue containing antibodies against the DPα:DQβ heterodimer are unsuitable to recipients having the DPα:DQβ heterodimer.

Methods of determining whether a donor tissue intended for transplant or transfusion is suitable for transplantation or transfusion to a human recipient having antibodies to a DPα:DQβ heterodimer comprising a) obtaining a sample of the tissue intended for transplant or transfusion in the human subject, b) contacting the sample with an antibody specific for DPα:DQβ heterodimer, and c) detecting the complex of a DPα:DQβ heterodimer and antibody specific for a DPα:DQβ heterodimer, wherein the presence of the complex indicates that the donor tissue is unsuitable for transplantation to the human recipient. In this case, the donor tissue containing DPα:DQβ heterodimer are unsuitable for recipients having antibodies to the DPα:DQβ heterodimer.

Also provided are kits for screening for antibodies that specifically bind to a DPα:DQβ heterodimer comprising a panel of solid-phase substrates in which a DPα:DQβ heterodimer is immobilized thereto. According to one embodiment kits are provided wherein the solid-phase substrate is a set of microparticles wherein each microparticle presents at least one DPα:DQβ heterodimer antigen. According to a further aspect kits are provided in which the solid phase substrate is a single antigen bead.

Also provided are kits for screening for DPα:DQβ heterodimer antigens comprising a panel of antibodies specific for a DPα:DQβ heterodimer. According to one embodiment the solid-phase substrate is a set of microparticles wherein each microparticle presents at least one DPα:DQβ heterodimer antibody. According to a further embodiment the solid phase substrate is a single antibody bead.

### BRIEF DESCRIPTION OF THE DRAWINGS

FIG. 1 depicts antibody reactivity of DRa associated proteins in EBV transformed B cell line 9066;

FIG. 2 depicts the differential surface expression of stable transfected LCL3023 with various DQβ1 allele and DRα. FJ5109 and L243 were used to detect Class II DQβ1 and DRα, respectively. Black dots represent the events from transfectants and grey dots represent the events from the LCL3023 host. Fluorescent intensity is depicted on the X axis and Side Scatter (SSC) is depicted on the Y axis;

FIGS. 3A and 3B show that DQβ:DRα proteins are immunogenic in human sera and depict examples of DQβ0601:DRα antibody reactivity in allosera to in relation to other HLA Class Il antibodies. DQβ0601:DRα and β0603:DRα proteins were attached to the non-overlapping Luminex beads and incorporated into the Class Il single antigen bead panel, LS2A01. Allosera that have high DQβ0601:DRα DRa antibody reactivity were tested on the bead pool using LABScreen assay protocol. The DQβ0601:DRα and β0603:DRα antibody reactivity were highlighted; and

FIGS. 4A-C depict the reactivity of DQβ0601:DRα antigen positive sera on DQβ0601:DRα transfectant cells. Three (3) DQβ0601:DRα positive sera with MFI of 28546 (4a), 85169 (4b) and 34889 (4c) on Luminex bead assay were tested on LCL3023 host transfected with DQB1*06:01 and DRA cDNA. Cells were stained with Biotinylated goat-anti-Human IgG followed by SAPE after serum incubation. A negative serum was also tested on the transfectant as the control.

FIG. 5 depicts the differential surface expression of stable transfectants on T2DM host with DQB1*0601 and various DPA1 cDNA clones. Alexa Fluor(R) 488 conjugated BRA-FB6 were used to detect DPA1* and Alexa Fluor(R) 594 conjugated HL-37 was used to detect DQB1*0601.

### DETAILED DESCRIPTION

Provided herein are methods of screening a biological sample for antibodies that bind to a DRα:DQβ heterodimer comprising the steps of:obtaining a biological sample from a subject, contacting the biological sample with a DRα:DQβ heterodimer, and detecting the binding of antibodies in said sample to the DRα:DQβ heterodimer. Similar methods are provided for DPα:DQβ heterodimers which can be produced by methods similar to those by which DRa:DQβ heterodimers are expressed. According to one aspect of the invention the subject is a mammalian subject and more preferably a human subject. Such methods are useful for determining whether a donor tissue intended for transplant or transfusion is suitable for transplantation or transfusion to a human recipient

Reported here in is a stable DQβ1/DRa heterodimer with cell membrane expression. DRA proteins from a homogeneous DRA EBV transformed cell line (9066) were purified via affinity columns conjugated with L243 (monoclonal antibody specific for DRA) and with FM5148 (monoclonal antibody specific for DQ).

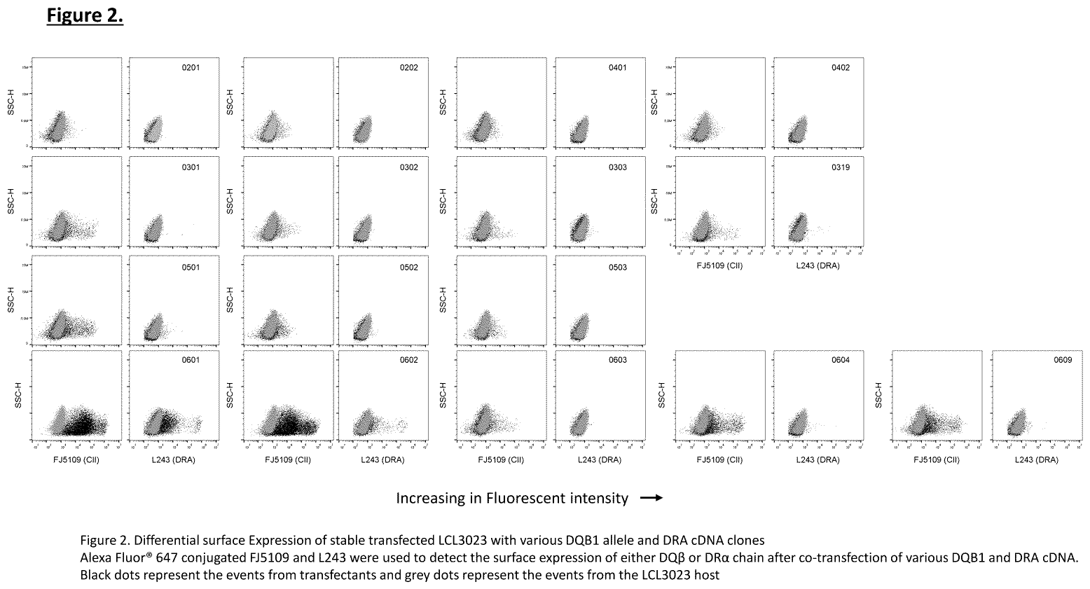

To further study the expression of the DQβ1/DRα heterodimer multiple DQβ1 alleles were co transfected with DRA in to LCL3023 (no Class Il genes) as shown in Table 2. These transfectants were tested by flow cytometry for the surface cell expression of Class II with the anti Class Il monoclonal antibody FJ5109 and anti-DRA L243 monoclonal antibody as shown in FIG. 2. DQβ1*06:01DRA and DQβ1*06:02DRA have the highest transfection efficiency and cell membrane expression followed by DQβ1*06:04 >DQβ1*06:09>DQβ1*05:01>DQβ1*03:01>DQβ1*03:19/ DRA heterodimers. These data show that multiple DQβ1/DRα heterodimers were able to be not only assembled intracellularly but transported to the cell membrane surface with differential expression.

To further characterize the DQβ1/DRα heterodimer expression purified protein from transfectants were bound to microbeads and tested against well-defined monoclonal antibodies, Table 3 clearly shows that there is a different reactivity between DQβ1*06:01/DRα and DQβ1*06:03/DRα suggesting that there is a different conformation or epitope recognition amongst these heterodimers by the reactivity to these well characterized monoclonal antibodies.

Microbeads bound with DQβ1*06:01DRα and DQβ1:06:03/DRaβ heterodimers were tested against 797 human sera (Table 4). Polyclonal human sera was able to react to these heterodimers and results showed that there is a similar frequency 5.1 vs 3.5% of these antibodies to these heterodimers respectively. This further confirms that heterodimers, even though at a lower frequency, can occur naturally as demonstrated by reactivity in polyclonal human sera.

DQβ1*06:01/DRα and DQβ1*06:03/DRα heterodimers were included as part of the Class II SAB panel, LSAA01. The human heterodimer antibody showed that it was very specific against the heterodimer alone and was negative to both DQβ1*06:01 and DQβ106:03 as well as individual DRα proteins from all DR antigens, suggesting that these antibodies reacted to a tertiary conformation between the DQβ1/DRachain potentially binding a different peptide than conventional DQβDQα dimers. On Class Il molecules, the peptide binding groove is made up of the α1 and β1 domains.

The antibody reactivity for DQβ:DRα heterodimers was defined as positive to only proteins that have both DQβ1 and DRα, but negative to the proteins that contain only one of the DQβ1 or DRα alleles and not both, so the antibodies were most likely reacting to the tertiary structure epitope composed of amino acids from both alpha and beta chains. In most of the HLA antibody epitope analysis algorithms, either linear or discontinuous sequences of a specific chain are used to assign the cross reactivity of an antibody. The identification of antibodies in patients that react to the heterodimers demonstrates the complexity of the interpretation HLA antibody specificity for potential donor selection.

Antibodies to non-isotypic class Il antigens further increase the challenge of identifying optimal donors.

The invention provides methods of detecting antibodies specific for DRa:DQβ and DPα:DQβ heterodimers. Preferably the DRα:DQβ and DPα:DQβ heterodimer antigens are presented on microbeads.

Exemplary solid-phase assays such as assays of the invention may use solid substrates such as microparticles, magnetic particles such as ferromagnetic beads and paramagnetic beads, microtiter plates, membranes, filters, glass, metal, metal-alloy, anopol, polymers, nylon, plastic or microarrays such as protein chips. Microarrays may be of any material such as glass or silica. Binding on a microtiter plate may be detected using ELISA assays, RIA assays or other immunosorbent sandwich assays. Binding on a filter may be detected using immunoblotting techniques.

The solid-phase assays of the invention may be carried out with microparticles, microbeads, magnetic beads, beads or microspheres of any material, e.g., silica, gold, latex, polymers such as polystyrene, polysulfone and polyethyl, or hydrogel. Additional exemplary microparticles are encoded with the dyes and the antigens are immobilized to the encoded microparticles. The microparticles used in the methods of the invention are commercially available from sources such from Luminex Inc., Invitrogen (Carlsbad, CA), Polysciences Inc. (Warrington, PA) and Bangs Laboratories (Fishers, IN) to name a few.

The microparticles of the invention may comprise a detectable label or another identifying characteristic. The microparticles may comprise a single fluorescent dye or multiple fluorescent dyes. In one embodiment, the microparticles are internally labeled with fluorescent dyes and contain surface carboxyl groups for covalent attachment of biomolecules. In another embodiment, the microparticles are internally labeled with fluorescent dyes and contain a surface layer of Avidin for near covalent binding of biotin and biotinylated ligands. In another embodiment, the microparticles may comprise a combination of different dyes, such as a fluorescent and a non-fluorescent dye. For example, the microparticles may be labeled with E)-5-[-[2-methoxycarbonyl)ethenyl]cytidine , which is a nonfluorescent molecule, that when subjected to ultraviolet (UV) irradiation yields a single product, 3-β-D-ribofuranosyl-2,7-dioxopyrido[2,3-d]pyrimidine, which displays a strong fluorescence signal. In another embodiment, the microparticles may comprise bar codes as an identifiable characteristic as described in U.S. Patent Publication No. 2007/0037195.

In another embodiment, the microparticles may be nanocrystals or quantum dots. These nanocrystals are substances that absorb photons of light, then re-emit photons at a different wavelength (fluorophores). In addition, additional florescent labels, or secondary antibodies may be conjugated to the nanocrystals. These nanocrystals are commercially available from sources such as Invitrogen and Evident Technologies (Troy, NY).

The invention can be carried out with any system that detects the identifiable characteristic or label, such as FLOW cytometry. Detection of fluorescent labels may also be carried out using a microscope or camera that will read the image on the microparticles, such as the Bioarray BeadChip (Bioarray Solutions, Ltd., (Immucor) Warren, NJ). The BeadChip format combines microparticle (“bead”) chemistry with semiconductor wafer processing in which binding to the microparticle is recorded using an optical microscope and camera.

The invention also may be carried out using column chromatography, affinity chromatography, thin layer chromatography, liquid-phase immunodiagnostic (LIPA) assays, liquid-phase chemiluminescent ELISA and liquid-phase immunoradiometric (IRMA) to name a few.

Biological samples include whole blood, blood derivatives, red blood cell concentrates, plasma, serum, fresh frozen plasma, whole blood derived platelet concentrates, apheresis platelets, pooled platelets, intravenous gamma-globulin, cryoprecipitate, cerebrospinal fluid, tissues and cells such as epithelial cells, such as those collected from the bucal cavity, stem cells, leukocytes, neutrophils and granulocytes. The biological samples may be obtained from a human donor of tissue or cells intended for transplantation or a human donor of blood or blood derivatives intended for transfusion. The biological sample may be obtained from a healthy bone marrow donor or a subject of a paternity test. The biological sample may also be obtained from a human subject that is an intended recipient of a transplant or transfusion, or the human subject that is donating the tissue or organ intended for transplantation or transfusion. Alternatively, the biological sample may be obtained directly from tissues or cells that are intended for transplantation in a human recipient. In addition, the biological sample may be obtained from blood or blood derivatives that are intended for transfusion in a human recipient.

The antibodies of the invention may be polyclonal antibodies, monoclonal antibodies, antibody fragments which retain their ability to bind their unique epitope (e.g., Fv, Fab and F(ab)2 fragments), single chain antibodies and human or humanized antibodies. Antibodies may be generated by techniques standard in the art using an antigenic HLA epitope. See, e.g., Kohler et al., Nature, 256:495-497 (1975), Brodeur et al., Monoclonal Antibody Production Techniques and Applications, pp. 51-63 (Marcel Dekker, Inc., New York, 1987). Antibody molecules of the present invention include the classes of IgG (as well as subtypes lgG 1, lgG 2a, and lgG2b), IgM, IgA, IgD, and IgE.

The antibodies of the invention may be labeled for detection of binding within the biological sample. The antibodies may comprise a radioactive label such as ³H, ¹⁴C, ³²P, ³⁵S, or ¹²⁵I. In addition, the labels may be a fluorescent or chemiluminescent compound, such as fluorescein isothiocyanate, phycoerythrin, rhodamine, or luciferin. The labels may be enzymes such as alkaline phosphatase, β-galactosidase, biotin and avidin or horseradish peroxidase (Bayer et al., Meth. Enz., 184:138-163 (1990)).

The antibodies specific for HLA antigens may be attached to solid substrates such as membranes, beads, filters, glass, silicon, metal, metal-alloy, anopore, polymeric, nylon or plastic for detection of antigens in a biological sample.

Specific binding of an antibody to an HLA antigen within a biological sample may be carried out using Western blot analysis with immunoblotting, immunocytochemistry, immunohistochemistry, dot blot analysis, flow cytometry, ELISA assays or RIA assays. These techniques and other approaches are conventional in the art (See Sambrook et al., Molecular Cloning:A Laboratory Manual, cold Springs Harbor Laboratories (New York, 1989).

Of interest to the present invention are assay methods making use of flow cytometry. Wilson et al., J. Immunol. Methods 107:231-237 (1988) disclose the use of polyacrylamide microspheres coupled with cell membrane proteins in immunofluorescence assays for antibodies to membrane-associated antigens. The method is said to make possible the rapid flow cytometric analysis of plasma membrane antigens from cell populations that would otherwise be unsuitable for use in flow cytometry. Scillian et al., Blood 73:2041-2048 (1989) disclose the use of immunoreactive beads in flow cytometric assays for detection of antibodies to HIV. Frengen et al., Clin. Chem. 40/3:420-425 (1994) disclose the use of flow cytometry for particle-based immunoassays of ce-fetoprotein (AFP). This reference further reports the ability of serum factors to cross-link labeled mouse monoclonal antibodies of irrelevant specificity to different particle types coated with various immunoglobulins.

Flow cytometry methods using lymphocytes are also known but suffer with difficulties because of the activity of auto-antibodies. See Shroyer et al., Transplantation 59:626-630. Moreover, when using flow cytometry with lymphocytes, use of ten or more different lymphocytes tends to result in confusing signals. As a consequence, studies using lymphocytes have been limited by presenting a small panel of HLA antigens that do not effectively simulate the distribution of HLA antigens in a normal human population.

The invention also provides for kits to carry out the methods of the invention. In particular, the invention provides for kit for determining the presence of antibodies in serum of a subject against HLA Class Il antigens comprising a first collection of solid-phase substrates wherein each solid-phase substrate is coated with different purified DRa/DQβantigens to represent the HLA antigen population of a single cell line such that said collection simulates the distribution of DRα/DQβ antigens in a normal human. The antigens provided in the kit may be conjugated to solid substrates in the kit. Alternatively, the kit comprises solid substrates and antigens and the skilled artisan can conjugate the antigens to the solid substrates allowing for optimization of the antigens used in the assay. The kits may also comprise the reagents necessary to detect and measure antibodies, such as HLA antibodies for use as a positive control.

The kits useful herein may further comprise any components necessary to carry out the detection assays that are conventional in the art. For example, the kits may comprise buffers, loading dyes, gels such as polyacrylamide gels and molecular weight markers for preparing SDS-PAGE gels to carry out Western blots. The kits may also comprise filters, membranes blocking buffers, control buffers, isotype control antibodies, wash buffers or buffers and reagents for detection to carry out immunoblotting or dot blotting analysis such as labeled secondary antibodies. The kit may also comprise fixing reagents, blocking buffers, control buffers, wash buffers, staining dyes and detection reagents including anti-idiospecific antibodies. Furthermore, the kits may comprise the necessary reagents and tools to carryout flow cytometry, ELISA assays, RIA assays or microtoxicity assays.

In some embodiments, the detecting step comprises detecting labeled ligand bound to the complex to determine the presence or absence of antibodies reactive against selected DRα/DQβ antigens. In some embodiments, detecting of the labeled ligand is carried out by flow cytometry. In some embodiments, the detecting step comprises detecting the presence of the complex using a solid phase immunoassay or a multiplexed bead immunoassay.

The solid-phase substrate can be any solid substrate known in the art. In some embodiments, the solid-phase substrate is selected from the group consisting of microparticle, microbead, magnetic bead, ion torrent bead, flow cytometer bead and an affinity purification column. In some embodiments, the solid-phase substrate is a microbead. In some embodiments, the microbead is a latex microbead. The microbead, in some embodiments, has a diameter ranging from about 2 um to about 15 μm, inclusive. Microbeads having a diameter of about 2 μm, 3 μm, 4 μm, 5 μm, 6 μm, 7 μm, 8 μm, 9 μm, 10 μm, 11 μm, 12 μm, 13 μm, 14 μm or 15 μm are also contemplated. In some embodiments, at least one microbead presenting Class I HLA antigens is 3 μm is diameter. In some embodiments, at least one microbead presented Class II HLA antigens is 5 μm in diameter. In some embodiments, the microbeads comprise a mixture of 3 μm microbeads presenting Class I HLA antigens and 5 μm microbeads presented Class II HLA antigens.

In some embodiments, each solid phase substrate is detectably distinguishable from other solid phase substrates within the composition. In some embodiments, the detectably distinguishable solid phase substrates are distinguishable by fluorescent labels.

### EXAMPLES

### Material and Methods

### Cell lines and antibodies:

International Histocompatibility Workshop and Conference cell lines 9062 (DRB1*13:01, DRB3*01:01, DQA1*01:03, DQβ1*06:03, DPA1*01:03, DPB1*04:01, DPB1*02:01) and 9066 (DRB1*08:03, DQA1*01:03, DQβ1*0601, DPA1*02:02, DPB1*02:02) (5) were kindly provided by Dr. Jennifer Ng. EBV transformed B cell line LCL3023 (3) were kindly provided by Dr. William Burlingham. T2 cell line (4) was purchased from ATCC. Both LCL3023 and T2 are derived from LCL 721.174 (3,4) which has a homozygous deletion from DRA to DPB1 and has no detectable expression of HLA Class Il on the surface of the cell membrane. Antibodies used in this study are listed in Table 1. Human sera were collected from patients in the waiting list with consent.

TABLE 1
Characteristics of the Antibodies used in this study

| Clone | Specificity | Type | Host | Source |
|---|---|---|---|---|
| 17221-1-AP | DRA | Polyclonal | Rabbit | Thermo Fisher Scientific, |
| | | | | Waltham, MA |
| PA5-52927 | DQA1*01 | Polyclonal | Rabbit | Thermo Fisher Scientific, |
| | | | | Waltham, MA |
| PA5-78003 | DQB | Polyclonal | Rabbit | Thermo Fisher Scientific, |
| | | | | Waltham, MA |
| HL-37 | DQβ | Monoclonal | Mouse | Thermo Fisher Scientific, |
| | | | | Waltham, MA |
| L243 | DRA | Monoclonal | Mouse | BioLegend, San Diego CA |
| FM5148 | DQ | Monoclonal | Mouse | One Lambda, West Hills, CA |
| FJ5109 | Class II | Monoclonal | Mouse | One Lambda, West Hills, CA |
| LN3 | DR, DP, partial DQ | Monoclonal | Mouse | BioLegend, San Diego CA |
| TU36 | DR | Monoclonal | Mouse | BioLegend, San Diego CA |
| TU39 | DR, DP, partial DQ | Monoclonal | Mouse | BioLegend, San Diego CA |
| TU169 | DQ | Monoclonal | Mouse | BioLegend, San Diego CA |

### Transfection and Cell Culture

The DQβ1 and DRA cDNA sequences were synthesized according to the published sequences in the IPD-IMGT/HLA Database (https://www.ebi.ac.uk/ipd/imgt/hla/). Each DQβ1 allele and DRA were cloned into the pEF6 Mammalian Expression Vector (Themo Fisher Scientific, Waltham, MA) and co-transfected into either T2 or LCL3023 human cell lines using a Neon Transfection System (ThermoFisher Scientific, Waltham, MA) according to the manufacturer's protocol. Cells were pulsed twice with a voltage of 1,100 and a width of 30. Stable cells were established by RPMI-1640 media with 10% FBS and 5 μg/ml Blasticidin-S (Thermo Fisher Scientific, Waltham, MA).

### HLA Antibody Assay

HLA Class Il antigens were purified by cell lysis and affinity purification from cell pellets (Ref.1). Purified antigens were attached to the microparticles (Ref 2). Solid-phase bead assay was performed as the LABScreen (TM) antibody detection protocol and analyzed by a LABScan3D multiplex flow analyzer (One Lambda, West Hills, CA). The basic protocol follows the following steps:

Add 5 μl of LABScreen beads with 20 μl of test serum

Incubate for 30 minutes in the dark at 20-25° C. with gentle shaking

Add 150 μl of 1X wash buffer and vortex

Centrifuge at 1,300 g for 5 minutes. Remove wash buffer

Add 200 μl of 1X wash buffer and vortex

Centrifuge at 1,300 g for 5 minutes. Remove supernatant

Repeat Steps 5 and 6

Add 100 μl of 1X PE-conjugated anti-human lgG and vortex

Incubate in the dark for 30 minutes at 20 - 25º C with gentle shaking

Centrifuge at 1,300 g for 5 minutes and remove supernatant

Repeat Steps 5 and 6 twice

Add 80 μl 1X PBS and vortex

Proceed to data acquisition and analysis using a LABScan 3D instrument

### Flow Cytometry Analysis and Cell Sorting

Cells were stained with a primary antibody conjugated with Alexa Fluor® 647 (#307621, BioLegend), for 30 min at room temperature. FC analyses were performed in an Quanteon Flow Cytometer (Agilent, Santa Clara, CA). Data was analyzed with FlowJo Software.

Stable transfected cells were sorted on a MA900 cell sorter (Sony Biotechnology, San Jose, CA). High immunofluorescent cells were sorted and plated in RPMI-1640 with 10% FBS and 5 ug/ml Blasticidin-S.

### Example 1

### DRα associated antigens in EBV transformed B cell line 9066 contain both DRB and DQβ Chains.

Since it has been reported that DRα chain forms dimers with not only the DRβ chain, but also DQβ chain, we hypothesized by purifying cell lysate from B cell lines with DRa-specific monoclonal antibody L243, we will obtain both the associated DRβ and DQβchains. HLA Class II Proteins were purified from the cell lysate of an EBV-transformed B-cell line 9066 via affinity columns conjugated either with a DRa-specific monoclonal antibody, L243, or a DQ-specific monoclonal antibody, FM5148.

The bound proteins were eluted from the columns and attached to microparticles and characterized with a panel of monoclonal antibodies with known specificity using Luminex bead assay protocol. The proteins purified by L243 column reacted to DRα and DRB specific monoclonal antibodies as expected for the DR0803 protein, however, it also reacted to the DQβ-specific monoclonal antibody, HL-37, but negative with the DQα-specific antibody, Tu169, indicating the presence of DQβ:DRα heterodimer. The proteins purified with DQ-specific monoclonal antibody reacted to three DQ specific monoclonal antibodies, FM5148, Tu169 and HL-37, but negative to two DR-specific antibodies, L243 and Tu36.

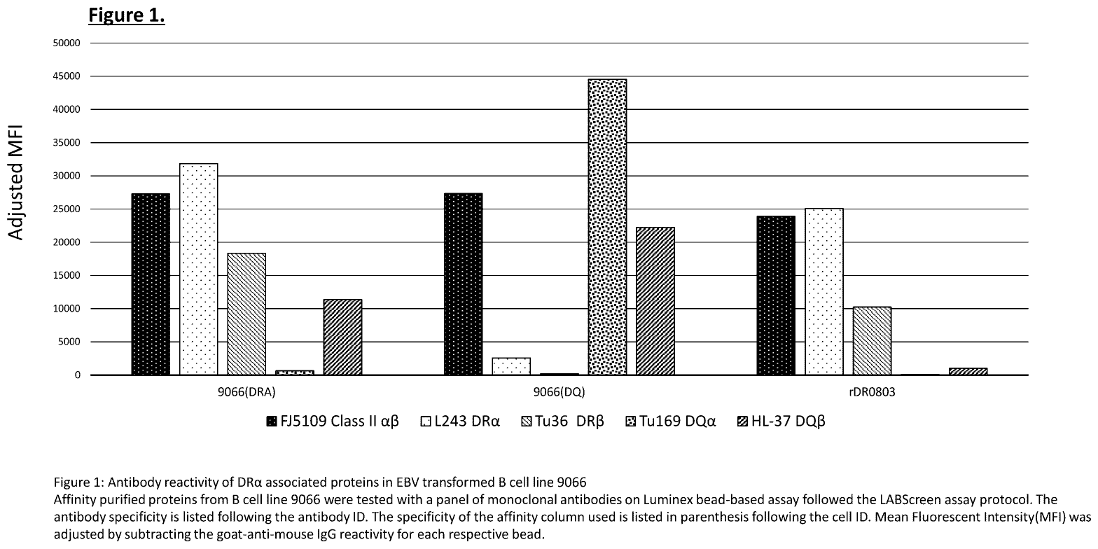

These results demonstrated that the DQβ0601 protein was purified via its dimerization with DRα chain and not as an impurity that co-purified with DRα from the L243 affinity column because no DQa1 protein was identified (FIG. 1).

### Example 2

### Co-transfection with Various DQβ1 Alleles and DRA cDNA to LCL3023 B Cell Line Produced DQβ:DRα Heterodimer Surface Expression with Different Efficiency

Plasmids with DRA and various DQβ1 alleles (Table 2) were constructed and used to transfect LCL3023, a cell line that doesn't express any Class II genes. Surface expression of DRα and DQβ1 on the transfectants were detected by flow cytometry using either PE-conjugated anti-Class II (FJ5109) or anti-DRA (L243) monoclonal antibody.

QB1*06:01DRA and DQβ1*06:02DRA plasmids have the highest transfection efficiency followed by DQβ1*06:04DRA, DQβ1*06:09DRA and DQβ1*05:01DRA plasmids, then DQβ1*03:01DRA and DQβ1*03:19DRA plasmids. The transfection efficiencies for the rest of DQβ1:02 and DQβ1*04 alleles with DRA were either very low or undetectable (FIG. 2).

TABLE 2
DQB1 alleles used for co-transfection with DRA
DQB1 allele

DQB1*02:01
DQB1*02:02
DQB1*04:01
DQB1*04:02
DQB1*03:01
DQB1*03:02
DQB1*03:03
DQB1*03:19
DQB1*05:01
DQB1*05:02
DQB1*05:03
DQB1*06:01
DQB1*06:02
DQB1*06:03
DQB1*06:04
DQB1*06:09

### Example 3 Characterization of DQβ1:DRα heterodimers by solid-phase immunoassay

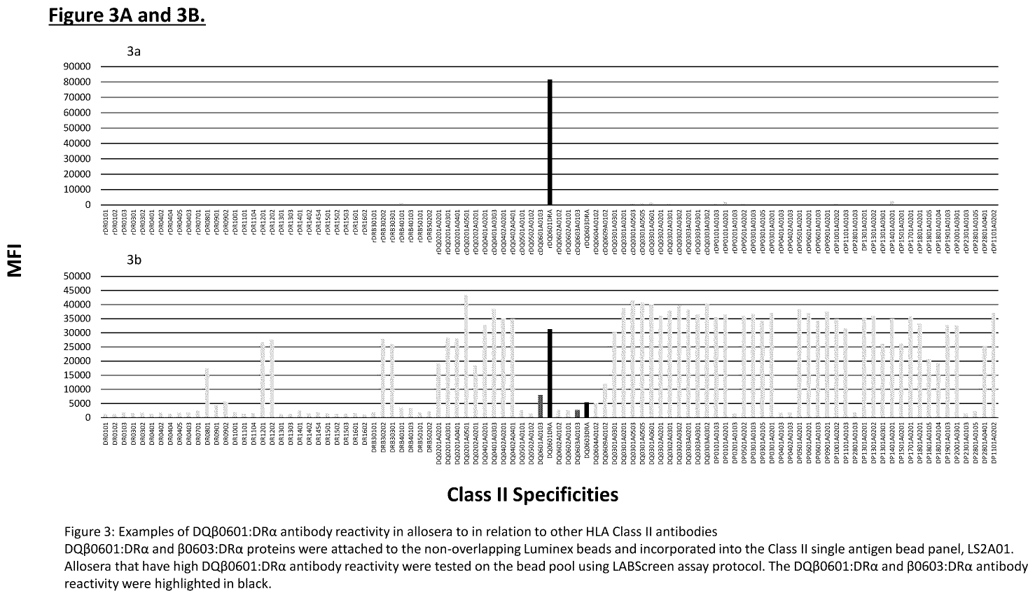

The reactivity of the purified DQβ0601:DQa0103 protein was distinctly different from other DQ6 antigens to different well-characterized monoclonal antibodies, as it was the only DQ6 protein that strongly reacted to the broad Class Il antibodies, LN3 and TU39 (FIGS. 3A and 3B) on the Class Il single antigen panel. Even though DQβ0603:DQa0103 protein shares the same DQa0103 chain and has very similar DQβ chain sequence, reacts negatively to both monoclonal antibodies.

The DQβ0601:DRα has a similar positive reactivity as the DQβ0601:DQa0103, indicating the epitope recognized by either LN3 or TU39 is located on the DQβ0601 chain and not on the DQa0103 chain. Conversely, both DQβ0601:DRα and DQβ0603:DRa heterodimers reacted negatively to the anti-DQ monoclonal antibody, TU169, confirming the epitope for TU169 is located on the DQα chain (Table 3).

TABLE 3
Comparison of antibody strength of DQαβ dimers and DQβDRα
heterodimers by fluorescence intensity

| | 9058 | | TC67-1A | 9066 | TC65-1 |
|---|---|---|---|---|---|
| | DQ0603A0103 | | DQ0603DRA | DQ0601A0103 | DQ0601DRA |
| L243 (DRA) | − | (944) | + (22809) | − (3277) | + (44953) |
| LN3 (DR, DP, | +/− | (8341) | − (3438) | + (46036) | + (49677) |
| partial DQ) | | | | | |
| Tu36 (DR) | − | (340) | − (1654) | − (932) | − (891) |
| Tu39 (DR, DP, | − | (2047) | − (1842) | + (41841) | + (44375) |
| partial DQ) | | | | | |
| Tu169 (DQ) | + | (41380) | − (1800) | + (42814) | − (918) |

Custom single antigen beads reacted with Class Il monoclonal antibodies and analyzed on LABScan 3D. The positive and negative reactivities were assigned. The MFI values were shown in (Table 3).

### DQβ1*06:01 Beta Chain Forms a Heterodimer with DRα In a EBV Transformed B Cell Line

DRα chain associated proteins were purified from an EBV-transformed B-cell line homozygous for DRA (9066) via affinity columns conjugated either with a DRa-specific monoclonal antibody, L243 or a DQ-specific monoclonal antibody, FM5148. The bound proteins were eluted from the columns and attached to microparticles and characterized with a panel of monoclonal antibodies with known specificity using Luminex bead assay protocol. The proteins purified by L243 column reacted to DRα and DRβ specific monoclonal antibodies as expected for the DR8 protein, however, it also reacted to the DQβ-specific monoclonal antibody, HL-37, but negative with the DQα-specific antibody, Tu169, indicating the presence of DQβDRα heterodimer. The proteins purified with DQ-specific monoclonal antibody reacted to three DQ specific monoclonal antibodies, FM5148, Tu169 and HL-37, but negative to two DR-specific antibodies, L243 and Tu36.

These results demonstrated that the DQβ1*06:01 protein was purified via its association with DRA and not as an impurity that co-purified with DRA from the L243 affinity column because no DQα1 protein was identified (Table 4).

TABLE 4

| | | Reactivity (MFI) | | |
|---|---|---|---|---|
| | | Cell | | |
| | | 9066 | | 9066 |
| | | Column | | |
| Monoclonal | Antibody | L243 | | FM5148 |
| Antibody | Specificity | DR8+ DQ0601DRA | | DQ0601A0103 |
| FJ5109 | Class II αβ | + | (40334) | + (39364) |
| L243 | DRα | + | (42324) | − (2541) |
| Tu36 | DRβ | + | (26595) | − (204) |
| FM5148 | DQ αβ | − | (30) | + (44601) |
| Tu169 | DQα | − | (520) | + (42058) |
| HL-37 | DQβ (DR8 | + | (14165) | + (33249) |
| | negative) | | | |
| LN3 | DR, DP, | + | (46487) | + (45304) |
| | partial DQ | | | |
| Tu39 | DR, DP, | + | (43483) | + (41114) |
| | partial DQ | | | |

### Example 4

### DQβ1:DRα Proteins are Immunogenic in Human Sera

Microbeads with either DQβ0601:DRα or DQβ0603:DRα heterodimers were tested against 797 human sera (Table 2). The results showed that 5.6% of the sera exhibited positive reactivity to DQβ0601:DRα heterodimer, but only 0.3% to DQβ0603:DRa heterodimer. The DQβ0601:DQa0103 dimer and DQβ0603:DQa0103 dimers demonstrated similar frequencies of positivity reacting with 5.1% and 3.5% of the sera samples, respectively. Interestingly, there are more sera with high MFI to DQβ0601:DRα antigen than to DQβ0603:DRα antigen (Table 2).

The DQβ0601:DRα and DQβ0603:DRα heterodimers were incorporated into the Class Il single antigen bead panel, LS2A01, and used to test those sera with high DQβ0601:DRα antibody reactivity. The DQβ0601:DRα specific antibody was verified as negative to both the DQβ chain from DQβ0601:DQa0103 antigen and the DRα chain from all DR antigens. The DQβ0601:DRα antibody reactivity were observed either as the only HLA Class II antibody (FIG. 3A) or with other DR, DQ and DP antibodies (FIG. 3B).

Table 5. Frequencies of DQO601DRA and DQO603DRA positive sera and their strength.

Positive reaction specific to DQβ1 and DRAα antigen is defined as minimum of 1500 MFI adjusted by subtracting negative control bead and is greater than 3-fold of the MFI from the same DQβ1 allele with DQA1*01:03 antigen.

TABLE 5
Table 5: Frequencies of DQ0601DRA and DQ0603DRA positive sera and their strength.
Positive reaction specific to DQB1*06:01 DRA antigen is defined as minimum
of 1500 MFI adjusted by subtracting negative control bead and is greater than
3-fold of the MFI from the same DQB1 allele with DQA1*01:03 antigen.

| | DQ0601DRA | DQ0601A0103 | DQ0603DRA | DQ0603A0103 |
|---|---|---|---|---|
| Positive | 45 | 41 | 2 | 28 |
| % (797 total) | 5.6 | 5.1 | 0.3 | 3.5 |
| >40,000 MFI | 3 | 3 | 0 | 1 |
| <40,000, >20,000 | 3 | 7 | 0 | 9 |
| MFI | | | | |
| <20,000, >10,000 | 5 | 6 | 0 | 1 |
| MFI | | | | |
| <10,000, >2,000 | 34 | 25 | 2 | 17 |
| MFI | | | | |

### Example 5

### The Strong DQβ0601:DRα Sera Recognized Antigens Expressed on the Cell Surface of DQβ0601:DRα Transfectant

Three sera that have the highest reactivity (MFI) to the purified DQβ0601:DRα beads were tested on the transfectants that expressed DQβ0601:DRα proteins. Serum RO that was negative on DQβ0601:DRα beads was served as the baseline channel-shift for the DQβ0601:DRα transfectant.

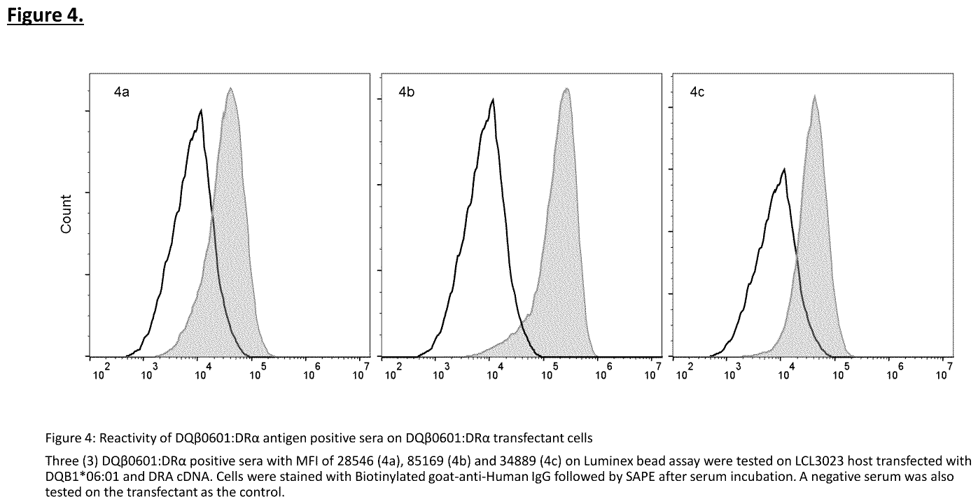

Serum #2 that has the highest MFI of 85169 on DQβ0601:DRα beads generated approximately 20-fold higher mean channel shift than the negative serum RO (FIG. 4b). Serum #3 with MFI of 34889 on DQβ0601:DRα beads generated approximately 4-fold higher mean channel shift than the negative serum RO (FIG. 4c).

To further understand the nature of the targeted epitopes for those DQβ0601:DRα positive sera, the same three sera were tested on the pH 2.7 treated beads and the reactivities were completely lost on all three sera suggesting these three sera recognize tertiary structure epitopes and not linear sequence epitopes (Table 3). In most of the HLA antibody epitope analysis algorithms, either linear or discontinuous sequences of a specific chain are used to assign the cross reactivity of an antibody. The identification of antibodies in patients that react to the heterodimers demonstrates the complexity of the interpretation HLA antibody specificity for potential donor selection.

TABLE 6
Reactivity (MFI) of DQβ0601:DRα positive
sera to the acid-denatured protein

pH 2.7 treatment

| Serum ID | No | Yes |
|---|---|---|
| #1 | +(25296) | −(436) |
| #2 | +(107417) | −(395) |
| #3 | +(35245) | −(306) |
| RO | −(264) | −(241) |

### Example 6

### Cross-Antigen Group Heterodimers Can Be Used to Assign Antibody Specificity to the Specific α or β Chains

According to this example the DRα:DQβ heterodimer with the uncommon association was used to distinguish the epitope is present on either the α or β chain. Normally HLA Class Il proteins are assembled by the a and B chains from the same Class (DR α and β, DQ α and β, DP α and β), so it is difficult to assign whether the antibody is recognizing the epitope on the α or β chain. However, a heterodimer such as DRα:DQβ, can be used to distinguish the epitope is present on either the α or β chain.

TABLE 7

| | LR13-1A | 3C65-5 | 9066 | Specificity |
|---|---|---|---|---|
| Antibody ID | DRβ0901:DRα | DQβ0601:DRα | DQβ0601:DQα0103 | assignment |
| L243 | +(43537) | +(47487) | −(2542) | DRα |
| QA19A44 | +(37998) | +(46318) | −(3273) | DRα |
| Tu36 | +(43315) | −(174) | −(204) | DRβ |
| Tu169 | −(169) | −(546) | +(42058) | DQα |
| HL-37 | −(586) | +(34669) | +(33249) | DQβ |

From the example above, the DQβ0601:DRα antigen from transfectant 3C65-5 can be used to locate the a or B chain-specific epitope of the DR or DQ antibodies. Both L243 and QA19A44 are positive to the antigens that have DRα chain even though they have different B chain, so the epitope for both L243 and QA19A44 can be assigned to the DRa chain.

Tu36 is positive to the DRB0901:DRα antigen, but negative to the DRα containing DQβ0601:DRα antigen, so the epitope for Tu36 is on DRB chain. Tu169 is positive to the DQβ0601:DQα0103 antigen, but negative to the DQβ0601 containing DQβ0601:DRα antigen, so the epitope for Tu36 is on DQβ chain. HL-37 is positive to both antigens that have DQβ0601 chain even though they have different a chain, so the epitope for HL-37 is located on DQβ chain.

### Example 7

### Co-Transfection of DQβ1*06:01 with Various DPA1 Alleles cDNA to T2DM B Cell Line Produced DQβ:DPα Heterodimer Surface Expression with Different Efficiency

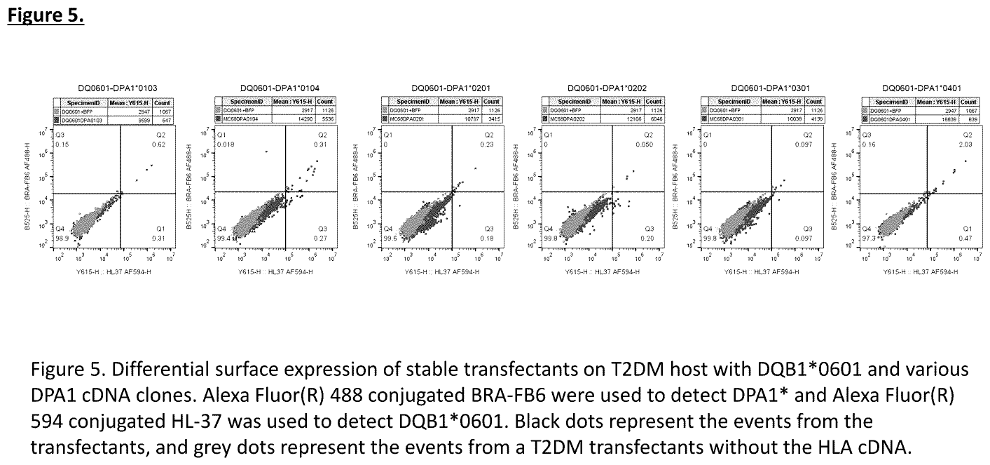

Plasmids with DQβ1*06:01 and each of the following DPA1 alleles:DPA1*01:03, DPA1*01:04, DPA1*02:01, DPA1*02:02, DPA1*03:01 and DPA1*04:01, were constructed and used to transfect T2DM, a B-cell line that doesn't express any Class Il genes, respectively. Surface expression of DPα and DQβ1 on the transfectants were detected by flow cytometry using either PE-conjugated anti-DQβ1 (HL-37) or anti-DPα (BRA-FB6) monoclonal antibody (FIG. 5). DQβ0601:DPα0401 has the highested co-expression efficiency (2.03%) followed by DQβ0601:DPα0103 (0.62%).

### Example 8

Characterization of DQβ0601:DPα0401 and DQβ0601:DPα0103 Heterodimers by Solid-Phase Immunoassay

The reactivity of the purified DQβ0601:DPα0401 and DQβ0601:DPα0103 proteins from the transfectants were characterized by different monoclonal antibodies that are specific to DQβ1 (HL-37), DQα1 (FM5148), DPα1 (BRA-FB6) or HLA-Class II (FJ5109) by solid-phase single antigen bead assay. Positivd reactions to HL-37 and BRA-FB6, and negative reaction to FM5148 confirmed the successful pairing of DQβ1 and DPα. Positive reactions to FJ5109 demonstrated the correct assembly of DQβ1 and DPα (Table 8).

The same DQβ0601:DPα0401 and DQβ0601:DPα0103 single antigen beads were used to screen sera from patients on the kidney transplant wait-list. Nine sera were identified to have positive reactivity (MFI>3000) to either or both DQβ:DPa single antigens but negative reactivity to the same DQβ chain not dimerized with DPa chain (DQβ0601:DQα0103) or the same DPα chain not dimerized with the DQβ chain (DPβ2801:DPα0401 or DPβ0401:DPα0103) demonstrating that the DQβ:DPα heterodimers are immunogenic (Table 9).

TABLE 8
Confirmation of the DQβ:DPα heterodimers
with locus-specific monoclonal antibodies

| | DQβ0601:DPα0401 | DQβ0601:DPα0103 |
|---|---|---|
| FJ5109 (HLA Class II) | + (25577) | + (18969) |
| HL-37 (DQB) | + (23325) | + (18916) |
| FM5148 (DQα) | − (2991) | − (2694) |
| BRA-FB6 (DPα) | + (20632) | + (18913) |

TABLE 9
Human sera that recognize only DQβ:DPα heterodimers and not individual α or β chain

| Serum ID | DQβ0601:DPα0401 | DQβ0601:DPα0103 | DQβ0601:DQα0103 | DPβ2801:DPα0401 | DPβ0401:DPα0103 |
|---|---|---|---|---|---|
| #4 | 4456 | 359 | 160 | 315 | 248 |
| #5 | 3192 | 908 | 172 | 211 | 152 |
| #6 | 4670 | 1272 | 209 | 241 | 224 |
| #7 | 1963 | 4474 | 259 | 302 | 250 |
| #8 | 1273 | 3739 | 250 | 405 | 402 |
| #9 | 1040 | 3315 | 285 | 355 | 291 |
| #10 | 1301 | 3563 | 240 | 385 | 393 |
| #11 | 1341 | 3364 | 160 | 251 | 222 |
| #12 | 3874 | 4338 | 337 | 293 | 352 |

Numerous modifications and variations in the practice of the invention are expected to occur to those skilled in the art upon consideration of the foregoing description on the presently preferred embodiments thereof. Consequently, the only limitations which should be placed upon the scope of the present invention are those that appear in the appended claims.

### References

Kao KJ:Plasma and platelet HLA in normal individuals:quantitation by competitive enzyme-linked immunoassay. Blood. 1987;70:282-286.

Pei R, Wang G, Tarsitani C, Rojo S, Chen T, Takemura S, Liu A, Lee JH: Simultaneous HLA class I and class Il antibodies screening with flow cytometry. Hum Immunol. 1998;59:313-322.

Ceman S, Rudersdorf RA, Petersen JM, DeMars R. DMA and DMB are the only genes in the class Il region of the human MHC needed for class Il-associated antigen processing. J Immunol. 1995;154:2545-2556.

Salter RD, Howell DN, Cresswell, P. Genes regulating HLA class I antigen expression in T-B lymphoblast hybrids. Immunogenetics. 1985;21:235-246.

Creary LE, Guerra SG, Chong W, el al. Next-generation HLA typing of 382 International Histocompatibility Working Group reference B-Lymphoblastoid cell lines:report from the 17th International HLA and Immunogenetics Workshop. Hum Immunol. 2019;80:449-460.

Lotteau V, Teyton L, Burroughs D, Charron D. A novel HLA class II molecule (DR alpha-DQ beta) created by mismatched isotype pairing. Nature 1987;329:339-341.

Lotteau V, Sands J, Teyton L, Turmel P, Charron D, Strominger JL. J Exp. Med. 1989;169:351-356.

Modulation of HLA class II antigen expression by transfection of sense and antisense DR alpha cDNA.

Kwok WW, Kovats S, Thurtle P, Nepom GT. HLA-DQ allelic polymorphisms constrain patterns of class II heterodimer formation. J Immunol. 1993;150:2263-2272.

What is claimed is:

1. A method of screening a biological sample for antibodies that bind to a heterodimer selected from the group consisting of DRα:DQβ and DPα:DQβ heterodimers comprising the steps of:
obtaining a biological sample from a subject,
contacting the biological sample with a heterodimer selected from the group consisting of DRα:DQβ and DPα:DQβ heterodimers, and
detecting the binding of antibodies in said sample to the heterodimer.

2. The method of claim 1 wherein the heterodimer is a DRα:DQβ heterodimer.

3. The method of claim 1 wherein the heterodimer is a DPα:DQβ heterodimer.

4. The method of claim 1 wherein the subject is a human subject.

5. The method of claim 1 wherein binding of an antibody is detected using flow cytometry.

6. The method of claim 1 wherein antibody binding is detected with a secondary antibody.

7. The method of claim 6 wherein the secondary antibody comprises a label selected from the group consisting of a radioactive label, fluorescent label, enzymatic label, colorimetric label avidin label and biotin label.

8. The method of claim 1 wherein the human subject is a transplant or transfusion recipient.

9. The method of claim 1 wherein the human subject is a transplant or transfusion donor.

10. A method of screening a biological sample for the presence of a heterodimer antigen selected from the group consisting of DRα:DQβ and DPα:DQβ heterodimer antigens comprising the steps of:
obtaining a biological sample from a subject,
contacting the biological sample with an antibody specific for said heterodimer, and
detecting the binding of said antibodies in said sample to a heterodimer selected from the group consisting of DRα:DQβ and DPα:DQβ heterodimers.

11. The method of claim 10 wherein the heterodimer is a DRα:DQβ heterodimer.

12. The method of claim 10 wherein the heterodimer is a DPα:DQβ heterodimer.

13. The method of claim 10 wherein the subject is a human subject.

14. The method of claim 10 wherein binding of an antibody is detected using flow cytometry.

15. The method of claim 10 wherein antibody binding is detected with a secondary antibody.

16. The method of claim 15 wherein the secondary antibody comprises a label selected from the group consisting of a radioactive label, fluorescent label, enzymatic label, colorimetric label avidin label and biotin label.

17. The method of claim 10 wherein the human subject is a transplant or transfusion recipient.

18. The method of claim 10 wherein the human subject is a transplant or transfusion donor.

19. A method of determining whether a donor tissue intended for transplant or transfusion is suitable for transplantation or transfusion to a human recipient expressing a heterodimer selected from the group consisting of DRα:DQβ and DPα:DQβ heterodimers comprising
a) obtaining a sample of the tissue intended for transplant or transfusion in the human subject,
b) contacting the sample with a heterodimer selected from the group consisting of DRα:DQβ and DPα:DQβ heterodimers, and
c) detecting the complex of a heterodimer selected from the group consisting of DRα:DQβ and DPα:DQβ heterodimers and antibody specific for heterodimer antigen selected from the group consisting of DRa:DQβ and DPα:DQβ heterodimers, wherein the presence of the complex indicates that the donor tissue is unsuitable for transplantation to the human recipient.

20. The method of claim 19 wherein the heterodimer is a DRα:DQβ heterodimer.

21. The method of claim 19 wherein the heterodimer is a DPα:DQβ heterodimer.

22. A method of determining whether a donor tissue intended for transplant or transfusion is suitable for transplantation or transfusion to a human recipient having antibodies specific for a heterodimer antigen selected from the group consisting of DRα:DQβand DPα:DQβ heterodimers comprising
a) obtaining a sample of the tissue intended for transplant or transfusion in the human subject,
b) contacting the sample with an antibody specific for a heterodimer antigen selected from the group consisting of DRa:DQβ and DPα:DQβ heterodimer, and
c) detecting the complex of a DRα:DQβ or DPα:DQβ heterodimer and antibody specific for a DRα:DQβ or DPα:DQβ heterodimer, wherein the presence of the complex indicates that the donor tissue is unsuitable for transplantation to the human recipient.

23. The method of claim 22 wherein the heterodimer is a DRα:DQβ heterodimer.

24. The method of claim 22 wherein the heterodimer is a DPα:DQβ heterodimer.

25. A kit for screening for antibodies that specifically bind to a DRα:DQβheterodimer comprising a panel of solid-phase substrates in which a heterodimer selected from the group consisting of DRα:DQβ and DPα:DQβ heterodimers is immobilized thereto.

26. The kit of claim 25 wherein the heterodimer is a DRα:DQβ heterodimer.

27. The kit of claim 25 wherein the heterodimer is a DPα:DQβ heterodimer.

28. The kit of claim 25 wherein the solid-phase substrate is a set of microparticles wherein each microparticle presents at least one a heterodimer antigen selected from the group consisting of DRα:DQβ and DPα:DQβ heterodimers antigens.

29. The kit of claim 25 in which the solid phase substrate is a single antigen bead.

30. A kit for screening for a heterodimer antigen selected from the group consisting of DRα:DQβ and DPα:DQβ heterodimers comprising a panel of antibodies specific for a heterodimer antigen selected from the group consisting of DRα:DQβ and DPα:DQβ heterodimer antigens.

31. The kit of claim 30 wherein the heterodimer is a DRα:DQβ heterodimer.

32. The kit of claim 30 wherein the heterodimer is a DPα:DQβ heterodimer.

33. The kit of claim 30 wherein the solid-phase substrate is a set of microparticles wherein each microparticle presents at least one antibody specific for a heterodimer selected from the group consisting of DRα:DQβ and DPα:DQβ heterodimers.

34. The kit of claim 30 in which the solid phase substrate is a single antibody bead.
